(19) United States
(12) Patent Application Publication
(10) Pub. No.: US 2014/0094780 A1
(43) Pub. Date: Apr. 3, 2014

(54) Wetting apparatus with releasable locked clamp

(71) Applicant: Medical Service GmbH, Bad Liebenzell (DE)

(72) Inventor: Bernhard HIESCH, Schomberg (DE)

(21) Appl. No.: 13/971,358

(22) Filed: Aug. 20, 2013

(30) Foreign Application Priority Data
Aug. 20, 2012 (DE) 102012016507.2

(51) Int. Cl. A61M 25/00

(52) U.S. Cl. CPC A61M 25/002
USPC 604/544

(57) ABSTRACT

A wetting apparatus for medical devices that can be inserted in the human body has a film pouch comprising a first chamber for accommodating the medical device and a second chamber in which a wetting agent is disposed, wherein the first chamber and the second chamber are separated from one another by a clamp attached to the outside of the film pouch. The wetting apparatus is an improvement on known devices, and facilitates a liquid-tight seal and ensures easy handling. To this end, the clamp comprises two interlocking strips between which the film pouch is enclosed, wherein one of the strips includes a groove that forms an undercut and the second strip forms a rib complementary to the groove.

The present invention relates to a wetting apparatus for medical devices that can be introduced into the human body and which has a film pouch comprising a first chamber for accommodating the medical device and a second chamber containing the wetting agent, wherein the first chamber and the second chamber are separated from one another by a clamp attached to the outside of the film pouch.

It is known to moisten the outer surface of medical instruments with a wetting agent in order to reduce sliding friction when introducing the medical device into hollows organ of the human body. The medical devices can then be introduced as pain-free as possible and without further irritations.

A typical application for such wetting apparatuses is, for example, one-time catheterization. When bladder function no longer occurs naturally, the urinary bladder must then be repeatedly emptied by means of thin disposable catheters. This process is also referred to as intermittent catheterization. In this process a thin disposable catheter is introduced through the urethra into the bladder and the urine is drained off through the catheter. Applying a lubricant to the catheter significantly reduces irritations of the urethra during insertion. Thus, it is known, for example, to coat catheters with a lubricant gel. In addition, there are catheters which have a hydrophilic coating that must be activated before the catheter is inserted in the urethra, for example, by moistening with water. Also now known for enabling simple catheterization en route are ready-to-use catheters which comprise a disposable catheter which is assembled together with the wetting agent in a packaging.

EP 1 982 741 A2, for example, shows a catheter set in which a catheter and the wetting agent for the catheter are assembled in a common packaging. The packaging comprises two chambers separated from each other by a weld seam. The catheter is situated in one of the chambers and the wetting agent in the other chamber. Prior to use the user presses on the chamber containing the wetting agent, the weld seam splits and the catheter is moistened.

Another ready-to-use urinary catheter set is described, for example, in EP 0 923 398 B1. The urinary catheter set comprises at least one partially hydrophilic coated urinary catheter and a catheter packaging that forms a cavity for accommodating the catheter. The catheter packaging further comprises a compartment that holds the wetting agent. The compartment is separated from the cavity that accommodates the catheter, the partition being designed such that it can be ruptured in order to moisten the catheter. It is shown, for example, that the compartment is formed by the packaging itself and is sealed by means of a clamp attached to the outside of the packaging and the walls of the catheter packaging opposite the cavity in which the catheter is located are pressed together.

This catheter set has the disadvantage that it is difficult for a user whose mobility is normally limited, to open the chamber containing the wetting agent. Moreover, the wetting agent must be securely located, separate from the catheter.

It is therefore the object of the present invention to provide a wetting apparatus which is a further improvement on the devices known from the prior art. In particular, a high degree of user-friendliness together with a secure, liquid-tight seal is to be achieved.

To this end the invention provides that the clamp comprise two interlocking strips between which the film pouch is enclosed, wherein one of the strips includes a groove that forms an undercut, and the second strip forms a rib complementary to the groove, the clamp being designed so that it opens as a result of a force exerted laterally to its longitudinal extension.

When the clamp is in the closed state, the walls of the film pouch are clamped between the two strips of the clamp. The second strip of the clamp with the rib is securely received in the groove of the first strip by virtue of the undercut. This prevents inadvertent opening. Moreover, a tighter seal is achieved. This allows for a seal of very simple design. For example, the clamp can be opened by the user grasping the film pouch on both sides of the clamp or on the ends of the film pouch and pulling on the film pouch. Another option for opening the clamp consists in the user squeezing together the second chamber containing the wetting agent, thereby compressing it, and exerting a pressure on the strips of the clamps. Thus, the second chamber that contains the wetting agent can be very easily opened even by a user with limited mobility, and the wetting agent brought into contact with the medical device to be wetted.

Clamp seals for films that comprise interlocking strips are in fact already known from DE 76 06 374 U and DE 72 02 949 U. However, DE 76 06 374 U relates to a packaging for a two-component adhesive used in the construction industry, in which the clamp is opened by pushing the strips apart. DE 72 02 949 U relates to the connection of two foil webs, for example, in the agricultural sector. A slider is provided for opening and closing the strips. The strips are designed to remain closed under transverse tensile stress.

The clip can further be advantageously designed so that it releases when a pressure applied to the second chamber containing the wetting agent exceeds 0.15 N/mm₂ (1.5 bar). A clamp thus designed prevents the second chamber containing the wetting agent from being inadvertently opened, but said second chamber is still easy for the user with limited mobility to open.

According to another embodiment the film pouch can exhibit a pressure resistance that amounts to twice the release force. Such pressure resistance applies both to any seal seams present in the film pouch as well as to the material of the film pouch itself. This ensures that the wetting agent remains in the film pouch when the wetting apparatus is activated, and does not escape outwardly.

According to still another advantageous embodiment the groove in the first strip and the rib of the second strip can have rounded contours. These rounded contours reduce the risk of the film pouch being torn when the clamp is opened and closed, and thus of the wetting agent leaking out.

To provide for a simple design and manufacture the first strip can be in the form of a tube with a slot extending in the longitudinal direction with the second strip designed as a rod that engages in the tube.

Furthermore, the clamp may be affixed to the film pouch. Thus, even after the clamp is opened, it remains appended to the film pouch, this facilitates easy handling and disposal of the wetting apparatus after each use.

In yet another embodiment the two strips of the clip can be articulately joined to one another at one end. Thus, the two strips remain connected to one another even after opening. This ensures resealability. In addition, both strips may remain attached to the film pouch after opening, thereby facilitating handling and disposal.

In this case, the clamp can be affixed to the film pouch in such a way that just one of the strips of the clamp is permanently attached to the film pouch.

In yet another embodiment the clamp may include an original seal which is destroyed during opening. With this original seal it is then possible to check prior to using the wetting apparatus whether the wetting apparatus is still in its original condition and the required sterility maintained.

The invention is described in greater detail below with reference to the drawings. In the drawings:

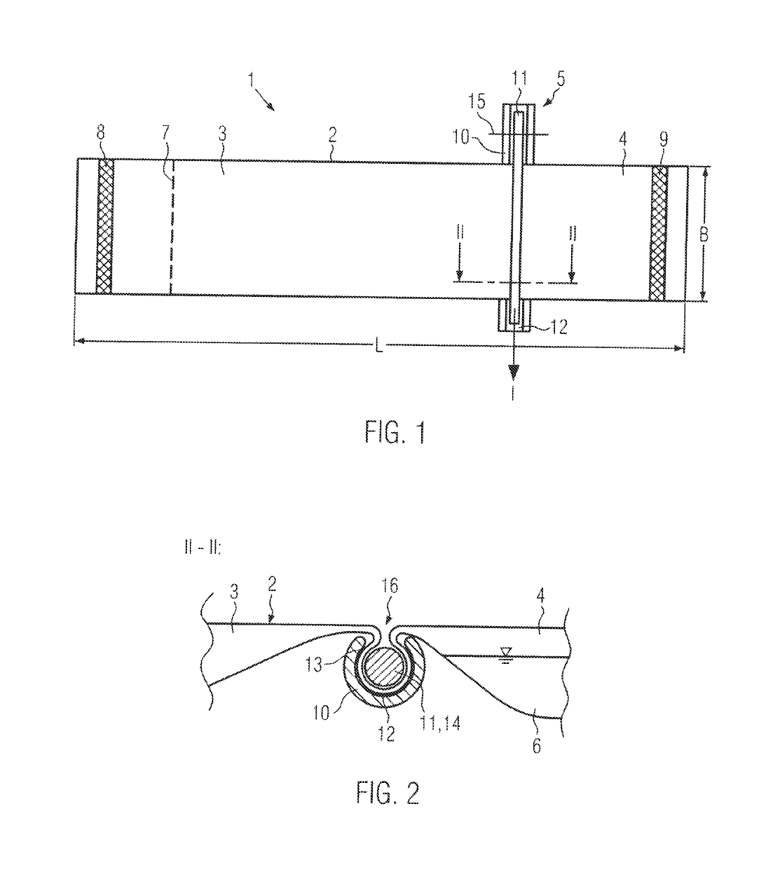

FIG. 1 shows a top view of a wetting apparatus according to the invention, and

FIG. 2 shows a section in expanded view through a portion of the wetting apparatus in FIG. 1 along the line II-II.

FIG. 1 shows a top view of a wetting apparatus 1 according to the invention. The wetting apparatus 1 comprises a film pouch 2, that forms a first chamber 3 and a second chamber 4. The first chamber 3 and the second chamber 4 are separated from one another by a clamp 5. The medical device, for example, a disposable catheter, is disposed in the first chamber 3. Disposed in the second chamber 4 is the wetting agent 6. The wetting agent 6 may, depending on the design of the catheter, be a lubricant gel or also a saline solution or sterile water. The film pouch 2 has a predetermined breaking point in the area of the first chamber 3.

At this predetermined breaking point the film pouch 2 can be easily opened and the medical device (not shown) removed. The predetermined breaking point 7 must be designed to be liquid tight.

In the case shown, the film pouch 2 is designed as a tubular plastic bag, with a longitudinal extension L that is significantly greater than its width B. The film pouch 2 is sealed in the longitudinal direction L at each end by a sealing seam 8, 9. Both the sealing seam 8 and the sealing seam 9 are designed to ensure a liquid-tight seal of the film pouch 2. However, a different design for the film pouch could also be envisaged. It is possible, for example, for the film pouch to consist of two layers made of a plastic film which are welded around their entire circumference by a liquid-tight seam seal.

The clamp 5 is defined by two strips 10, 11. The first strip 10 includes a groove 12 that forms an undercut 13. The second strip 11 forms a rib 14 in which the shape of the rib 15 corresponds approximately to the shape of the groove 12. Hence, the rib 14 is designed complementary to the groove 12. The first strip 10 and the second strip 11 are connected to one another at one end by a joint 15. At least one of the two strips 10, 11 is permanently attached to the film pouch 2, for example, by adhesion. However, it is also possible for both strips to be attached to the film pouch.

FIG. 2 shows a section in expanded view through the film pouch 2 with the clamp 5 attached therein along the line II-II in FIG. 1. The first strip 10 is in the shape of a circular tube and is provided with a slot 16 in the longitudinal direction of the clamp 5. The second strip 11 is designed as a rod with a circular cross-section and is arranged in the interior of the first strip 10. The slot 16 forms the undercut 13 that fixes the second strip 11 in the first strip 10. In the example shown, the first strip 10 is attached to the outside of the bottom of the film pouch 2. The second strip 11 is attached to the outside of the opposite side of the film pouch 2, that is, to the top of the film pouch 2. The film pouch 2, or the walls of the film pouch 2 are situated between the first strip 10 and the second strip 11. With the clamp 5 in the closed state, when the second strip 11 is snapped into the first strip 10 and fixed therein by the undercut 14, the side walls of the film pouch 2 are clamped between the first strip 10 and the second strip 11. This forms a liquid-tight seal. As shown in FIG. 2, the film pouch 2 starting from the first chamber 3 extends in the direction of the clamp 5, where it is bent sharply and clamped between the first strip 10 and the second strip 11. The tubular bag 2 follows the contours of the first strip 10 and of the second strip 11 and is guided approximately circularly in the clamp 5. In the direction of the second clamp 4, the film pouch 2, or the walls of the film pouch 2 are again bent sharply. This also ensures the liquid tightness of the seal. The contours of the first strip 10 and the second strip 11 are rounded to avoid damage to the film pouch 2.

The clamp is designed to open when a force is applied transversely to its longitudinal extension 1. The force applied may be a pulling force on both sides of the film pouch 2 to the left and right of the clamp 5. However, the force can also be applied by compressing the second chamber 5 that contains the wetting agent 6. The clamp is designed in such a way that it releases when a pressure applied to the second chamber 4 containing the wetting agent 6 exceeds a value of 0.15 N/mm₂ (1.5 bar). As a result of the pulling or pressure force effected thereby, the walls of the film pouch 2 are pulled outwardly thereby moving the second strip 11 in the direction of the slot 16. To enable the clamp 15 to release as desired, the width of the slot 16 should equal at least ¾ the diameter of the strip 11.

The use of the wetting apparatus 1 is described in the following. To wet the medical device disposed in the wetting apparatus 1, the user can grasp the film pouch 2 at both ends and pull the ends apart. The film pouch 2 situated between the strips 10, 11 then moves the second strip 11 outwardly, out of the first strip 10. This process is continued until the clamp 5 is completely open. Since the strips 10, 11 are connected to one another by the joint 15, they remain appended to one another in the open state. At least one of the strips 10, 11 is permanently attached to the film pouch 2 such that even in the open state the entire clamp 5 still remains attached to the film pouch 2. Once the clamp 5 is completely opened, the wetting agent 2 is able to flow from the second chamber 4 into the first chamber 3 where it wets the medical device, for example, a disposable catheter, disposed therein.

Alternatively, in order to open the clamp 5, the user can compress with one hand the second chamber 4 of the film pouch 2 which contains the wetting agent 6. By this means as well, the film pouch 12[sic] disposed between the two strips 10, 11 is pulled outwardly and the second strip 11 is leveraged out of the first strip 10, thereby opening the clamp 5. The wetting agent 6 then flows into the first chamber 3 and wets the medical device disposed therein. After wetting or activation of the medical device, the film pouch 2 may then be opened at the predetermined breaking point 7 and the medical device removed.

1. A wetting apparatus (1) for a medical device that can be introduced into the human body, which has a film pouch (2) comprising a first chamber (3) for accommodating the medical device and a second chamber (4) in which a wetting agent (6) is disposed, wherein the first chamber (3) and the second chamber (4) are separated from one another by a clamp (5) attached to the outside of the film pouch (2), wherein the clamp (5) comprises two interlocking strips (10, 11) between which the film pouch (2) is enclosed, wherein one of the strips (10) includes a groove (12) that forms an undercut (13) and the second strip (11) forms a rib (14) that is complementary to the groove (12), and wherein the clamp (5) is designed to be opened by a force applied transversely to its longitudinal extension (I).

2. The wetting apparatus according to claim 1, wherein the clamp (5) is designed so that it releases when a force applied to the second chamber (4) containing the wetting agent (6) exceeds 0.15 N/mm₂.

3. The wetting apparatus according to claim 1, wherein the film pouch exhibits a pressure resistance that amounts to at least twice the release force.

4. The wetting apparatus according to claim 1, wherein the groove (12) of the first strip (10) and the rib (14) of the second strip (11) have rounded contours.

5. The wetting apparatus according to claim 1, wherein the first strip (10) is shaped as a tube with a slot (16) extending in the longitudinal direction, and the second strip (11) is shaped as a rod that engages in the tube.

6. The wetting apparatus according to claim 1, wherein the clamp 5 is affixed to the film pouch (2).

7. The wetting apparatus according to claim 1, wherein both strips (10, 11) of the clamp (5) are articulately joined to one another at one end.

8. The wetting apparatus according to claim 6 wherein one of the strips (10, 11) of the clamp (5) is permanently attached to the film pouch (2).

9. The wetting apparatus according to claim 1, wherein the clamp (5) has an original seal which is destroyed when the clamp (5) is opened.
